(19) United States
(12) Patent Application Publication
(10) Pub. No.: US 2022/0125681 A1
(43) Pub. Date: Apr. 28, 2022

(54) MEDICAMENT COMPOUNDING DEVICES, SYSTEMS, AND METHODS

(71) Applicant: Chad Jensen, Hollister, MO (US)

(72) Inventor: Chad Jensen, Hollister, MO (US)

(21) Appl. No.: 17/080,606

(22) Filed: Oct. 26, 2020

(51) Int. Cl. A61J 1/20; A61J 1/14; A61J 1/06; A61K 33/00

(52) U.S. Cl. CPC A61J 1/2089; A61J 1/1406; A61J 1/065; A61K 33/00; A61J 1/201; A61J 1/2068; A61J 1/1412

(57) ABSTRACT

Devices, systems, and methods used to compound a first medicament and second medicament are disclosed. The devices include a single-use or single-dose vial containing a first medicament and configured to receive an ampule containing a second medicament. The vial includes a stopper and needle configured to inject the first medicament into the ampule to compound with the second medicament.

### TECHNICAL FIELD

The present disclosure relates generally to devices used to compound medicaments. More specifically, the present disclosure relates to a vial used to inject a first medicament into an ampule containing a second medicament. More specifically, the present disclosure relates to a vial used to inject a buffering medicament into an ampule containing an anesthetic medicament to change a pH value of the anesthetic medicament.

### BRIEF DESCRIPTION OF THE DRAWINGS

The embodiments disclosed herein will become more fully apparent from the following description and appended claims, taken in conjunction with the accompanying drawings. These drawings depict only typical embodiments, which will be described with additional specificity and detail through use of the accompanying drawings in which:

FIG. 1 is a perspective view of an embodiment of a medicament compounding vial.

FIG. 2 is a longitudinal cross-section of the medicament compounding vial of FIG. 1.

FIG. 3A is longitudinal cross-section of the medicament compounding vial of FIG. 1 with an ampule in a pre-compounding state.

FIG. 3B is a longitudinal cross-section of the medicament compounding vial of FIG. 1 with an ampule in an injected state.

FIG. 4 is a perspective view of another embodiment of a medicament compounding vial with a flange.

FIG. 5 is a longitudinal cross-sectional view of another embodiment of a medicament compounding vial with a vent needle.

### DETAILED DESCRIPTION

In certain instances, compounding of medicaments is a common practice performed by health care and dental care practitioners. The compounding may combine two or more medicaments for a single infusion or injection into a patient. In other embodiments, the compounding may combine two or more medicaments to modify a chemical property of one of the medicaments. In one embodiment, a buffering medicament, such as sodium bicarbonate, can be compounded with a local anesthetic medicament, such as lidocaine, to increase the pH of the lidocaine. The pH modified lidocaine may reduce injection-site pain and increase anesthetic onset. Thus, patient comfort may be improved and a duration of a procedure, such as a dental procedure, may be reduced. In some instances, a multi-use or multi-dose vial of the sodium bicarbonate may be used for a single use or patient resulting in higher procedural expenses and complexity.

Embodiments herein describe devices, systems, and methods to assist in compounding of medicaments just in time for use and at a point of care using a single use or single dose vial. In some embodiments, a single use or single dose medicament compounding vial can include a removable cap coupled to a housing. The housing defines a chamber divided by a moveable stopper. A single dose of a first medicament is contained within a portion of the chamber between the stopper and an end wall of the chamber. A needle is disposed through the stopper and is in fluid communication with the first medicament. In another embodiment, a vent needle is disposed through the stopper and is in fluid communication with a vent chamber of the housing.

In use, in certain embodiments, the cap is removed from the housing of the medicament compounding vial. An ampule containing a second medicament is disposed through an open end of the housing into the chamber. The needle punctures a septum of the ampule. A distally directed force is applied to the ampule such that the ampule distally displaces the stopper. The first medicament is pressurized by the stopper and flows through the needle into the ampule and is compounded with the second medicament within the ampule. In another embodiment, when the first medicament flows into the ampule the second medicament is pressurized and flows through the vent needle into the vent chamber.

As used herein, the terms “compound” and “compounding” refer to combining or mixing two or more medicaments together to form a medicament solution. In one embodiment, a first medicament may compound with a second medicament to modify or change a characteristic of the second medicament.

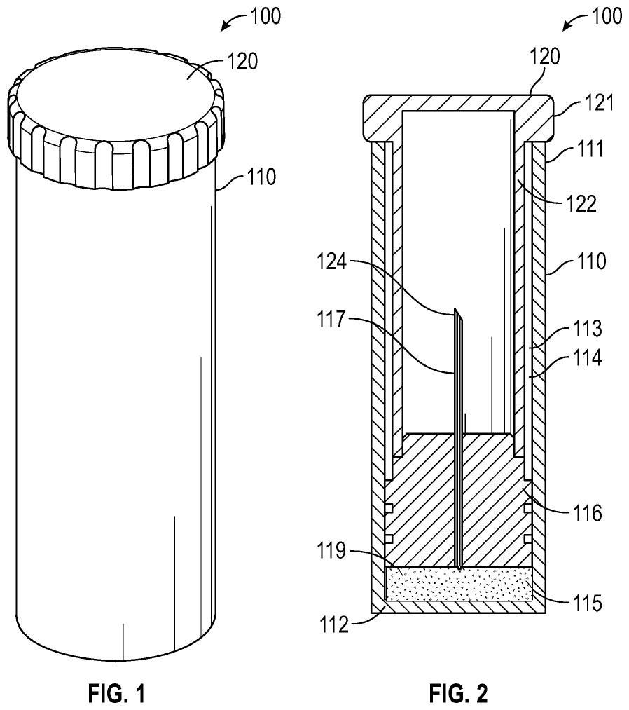

FIGS. 1-5 illustrate different views of medicament compounding vials and related components. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure or embodiment.

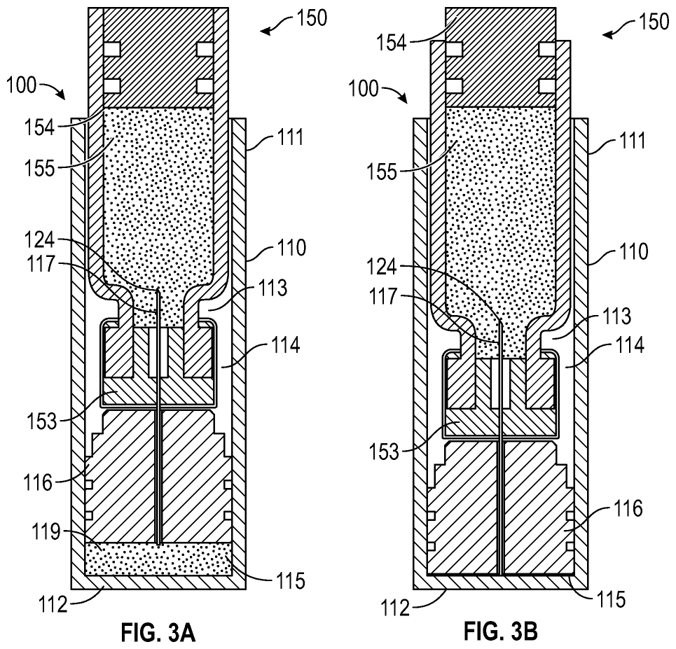

FIGS. 1-3B depict one embodiment of a medicament compounding vial 100. FIG. 1 is a perspective view of an embodiment of the medicament compounding vial 100. FIG. 2 is a longitudinal cross-section of the medicament compounding vial 100 of FIG. 1. FIG. 3A is longitudinal cross-section of the medicament compounding vial 100 of FIG. 1 with an ampule in a pre-compounding state. FIG. 3B is a longitudinal cross-section of the medicament compounding vial 100 of FIG. 1 with an ampule in an injected state. As illustrated in FIG. 1, the medicament compounding vial 100 includes two broad groups of components; each group may have numerous subcomponents and parts. The two broad component groups are: a housing 110 and a removable cap 120.

FIG. 2 shows a cross-sectional view of the medicament compounding vial 100. The housing 110 can include a proximal or open end 111, a distal or closed end 112, and a chamber 113 defined by the housing 110 and disposed between the open end 111 and the closed end 112. In the depicted embodiment, the housing 110 has a transverse cross-sectional circular shape. In other embodiments, the housing 110 may have any suitable transverse cross-sectional shape, such as square, oval, elliptical, hexagonal, etc. The housing 110 may be formed of any suitable material, such as glass, polycarbonate, acrylic, polypropylene, polyethylene, etc. using any suitable technique, such as glass blowing, injection molding, thermoforming, etc.

A stopper 116 can be disposed within the chamber 113 adjacent to the closed end 112. The stopper 116 may divide the chamber into a first or proximal portion 114 configured to receive an ampule 150 (as will be discussed below related to FIG. 3A) and a second or distal portion 115 configured to contain a first medicament 119. The stopper 116 may be configured to seal the distal portion 115 to retain the first medicament 119 within the distal portion 115 prior to use and to be slidably coupled to the housing 110. The stopper 116 can be formed of any suitable compliant material. For example, the stopper 116 may be formed of rubber, neoprene, thermoplastic elastomer, etc.

With further reference to FIG. 2, a first or injection hollow needle 117 can be disposed within the chamber 113 and oriented parallel with a longitudinal axis of the housing 110. The needle 117 may be disposed through the stopper 116 such that a distal end of the needle 117 is in fluid communication with the distal portion 115 of the chamber 113. A proximal portion of the needle 117 can extend from the stopper 116 within the proximal portion 114 of the chamber 113 toward the open end 111 of the housing 110. The needle 117 may include a penetration tip 124 disposed at a proximal end of the needle 117.

The first medicament 119 disposed within the distal portion 115 of the chamber 113, can include any liquid medicant compoundable with a second medicament. In one embodiment, the first medicament 119 may be a buffering agent, such as sodium bicarbonate. A concentration of the sodium bicarbonate can range from about 1% to about 10%, from about 4% to about 9%, and may be about 8.4%. A volume of the sodium bicarbonate may range from about 0.1 ml to about 1.0 ml and may be about 0.15 ml. In other embodiments, the first medicament can be any one of clonidine, dexmedetomidine, buprenorphine, dexamethasone, tramadol, midazolam, magnesium, epinephrine, morphine, fentanyl, and any combination thereof.

As shown in FIG. 2, the cap 120 can be removably disposed adjacent to the open end 111 of the housing 110 and may include a head portion 121 and a shaft portion 122. The cap 120 may be formed of any suitable material, such as glass, polycarbonate, acrylic, polypropylene, polyethylene, etc. using any suitable technique, such as glass blowing, injection molding, thermoforming, etc. The head portion 121 may be disposed exterior to the housing 110 and can be configured to be grippable by fingers of a user. In the illustrated embodiment, a diameter of the head portion 121 can be larger than an outer diameter of the housing 110. Additionally, the head portion 121 may include ribs and grooves disposed around a perimeter of the head portion 121 to facilitate gripping of the head portion 121. In other embodiments, the head portion 121 may include any suitable grippable feature, such as bumps, divots, texturing, over molding of a compliant material, etc.

The shaft portion 122 may be a cylinder with an open distal end and extend distally from the head portion 121 into the chamber 113. In the depicted embodiment, the distal end of the shaft portion 122 can be sized to frictionally fit with the stopper 116 to selectively couple the cap 120 with the stopper 116. When the cap 120 is removed from the housing 110, the head portion 121 may be gripped by fingers of the user and displaced proximally with or without rotation of the cap 120. In other embodiments, the head portion 121 and/or the shaft portion 122 may be selectively coupled with the housing 110. When the cap 120 is coupled to the housing 110 the proximal portion of the needle 117 can be disposed within the shaft portion 122. For example, in a certain embodiment, the head portion 121 can include internal threads that threadingly engage with external threads of the housing 110. In another embodiment, the shaft portion 122 may include external threads that threadingly engage with internal threads of the housing 110.

FIG. 3A depicts the medicament compounding vial 100 in a pre-compounding state. In the depicted embodiment, the cap 120 has been removed from the housing 110. A glass ampule or carpule 150 is disposed through the open end 111 of the housing 110 and into the proximal portion 114 of the chamber 113. The penetration tip 124 of the needle 117 has penetrated a septum 153 of the ampule 150 such that the needle 117 is in fluid communications with a second medicament 155 disposed within the ampule 150. In some embodiments the second medicament 155 may be a local anesthetic, such as lidocaine, articaine, bupivacaine, carbocaine, marcaine, mepivacaine, prilocaine, etc., and any combination thereof. In other embodiments, the second medicament 155 can be any desired medicament to be compounded with the first medicament 119. For example, the second medicament 155 may be clonidine, dexmedetomidine, buprenorphine, dexamethasone, tramadol, midazolam, magnesium, epinephrine, morphine, fentanyl, etc., and any combination thereof.

FIG. 3B depicts the medicament compounding vial 100 transitioned from the pre-compounding state, as shown in FIG. 3A, to a compounded state where the first medicament 119 is injected into the ampule 150 to compound with the second medicament 155. In the depicted embodiment, the closed end 112 of the housing 110 may be placed on a firm, flat surface and a distally directed force may be applied to the ampule 150. The ampule 150 may engage with the stopper 116 and apply a distally directed force to the stopper 116. The stopper 116 may be displaced distally resulting in pressurization of the first medicament 119 disposed within the distal portion 115 of the chamber 113. When the first medicament 119 is pressurized, the first medicament 119 may flow through the needle 117 and into the ampule 150 where it can be compounded or mixed with the second medicament 155. An ampule stopper 154 may be displaced proximally when the first medicament 119 flows into the ampule 150 due to the non-compressibility of the first and second medicaments 119, 155, respectively, and the rigidity of the ampule 150. The ampule stopper 154 can be displaced from about 1 mm to about 4 mm. When the first and second medicaments 119, 155, respectively, are compounded, the ampule 150 can be removed from the medicament compounding vial 100 and loaded into a re-usable ampule syringe holder.

When the first and second medicaments 119, 155, respectively, are compounded, the ampule 150 can be removed from the medicament compounding vial 100 and loaded into a re-usable ampule syringe holder. The compounded medicaments may be injected into a patient. In a certain embodiment, the first medicament 119 can be about 0.15 ml of an 8.5% solution of sodium bicarbonate and the second medicament 155 can be about 1.7 ml to about 1.8 ml of a 2% solution of lidocaine. When compounded, the sodium bicarbonate solution may modify the pH of the lidocaine solution from about 3 to 5 to closer to physiologic pH or 7. When the pH of the lidocaine solution is modified, the lidocaine solution may have increased bioavailability. Additionally, injection-site pain and local anesthesia onset time may be reduced resulting in increased patient comfort and reduced procedural times, such as dental procedures.

In some embodiments, the compounding of the first and second medicaments 119, 155, respectively, may occur approximately at a procedure start time and a point of care. This may ensure that a compounded solution does not change from the time of compounding to the time of delivery to a patient. For example, a shelf life of the compounded solution of sodium bicarbonate and lidocaine is short and the pH of the compounded solution will change from a physiologic pH to an acidic pH in a short period of time. Thus, the compounded solution of sodium bicarbonate and lidocaine should be delivered to the patient as close to a time of compounding as possible.

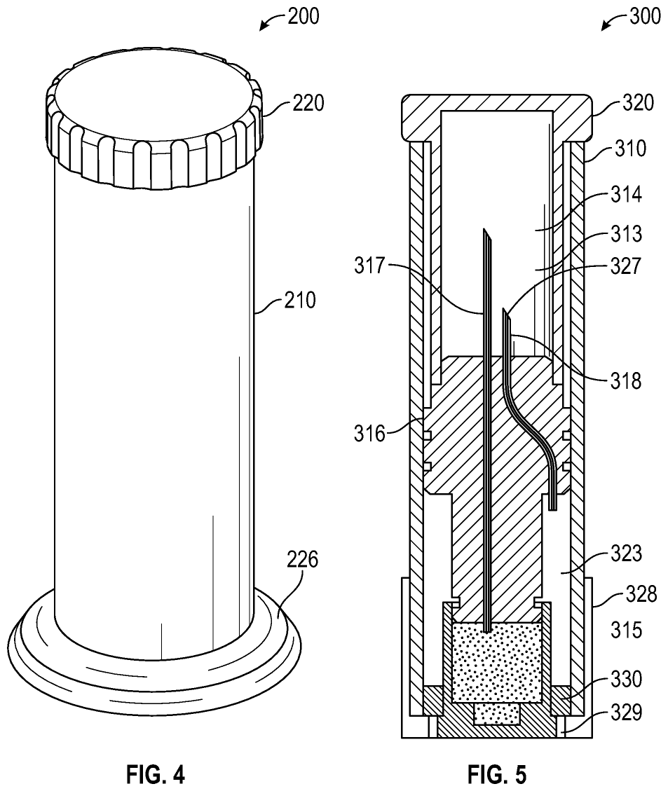

FIG. 4 depicts an embodiment of a medicament compounding vial 200 that resembles the medicament compounding vial 100 described above in certain respects. Accordingly, like features are designated with like reference numerals, with the leading digit incremented to “2.” For example, the embodiment depicted in FIG. 4 includes a housing 210 that may, in some respects, resemble the housing 110 of FIG. 1. Relevant disclosure set forth above regarding similarly identified features thus may not be repeated hereafter. Moreover, specific features of the medicament compounding vial 100 and related components shown in FIGS. 1-3A may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the medicament compounding vial 200 and related components depicted in FIG. 4. Any suitable combination of the features, and variations of the same, described with respect to the medicament compounding vial 100 and related components illustrated in FIGS. 1-3A can be employed with the medicament compounding vial 200 and related components of FIG. 4, and vice versa. This pattern of disclosure applies equally to further embodiments depicted in subsequent figures and described hereafter, wherein the leading digits may be further incremented.

FIG. 4 is a perspective view of another embodiment of the medicament compounding vial 200 and a flange 226. As illustrated in FIG. 4, the medicament compounding vial 200 includes a housing 210 and a cap 220. The housing 210 can include the flange 226 disposed at a distal end of the housing 210. The flange 226 can extend radial outwardly from the housing 210 to provide a broad support base when the medicament compounding vial 200 is placed on a firm, flat surface during use. As shown in the depicted embodiment, the flange 226 can include a circular shape. In other embodiments, the flange 226 may include any suitable shape configured to provide support to the medicament compounding vial 200. For example, the flange 226 may include a shape that is elliptical, oval, square, hexagonal, etc. In some embodiments, the flange 226 may be an integral part of the housing 210. In certain embodiments, the flange 226 can be a separate component assembled to the housing 210 using any suitable technique, such as friction fit, snap fit, bonding, welding, etc.

FIG. 5 is a longitudinal cross-sectional view of another embodiment of a medicament compounding vial 300 with a vent needle 318. As shown in FIG. 5, the medicament compounding vial 300 can include a housing 310. The housing 310 can include a vent chamber 323 disposed distally of a stopper 316. The vent chamber 323 may include an end cap 328. The end cap 328 may include a vent channel 329 to vent the vent chamber 323 through the end cap 328. A fluid absorbent mat 330 can be disposed within the vent chamber 323 and configured to absorb fluid, such as a medicament.

As illustrated in FIG. 5, the medicament compounding vial 300 may include a second or vent needle 318 disposed through a stopper 316 and in fluid communication with both the vent chamber 323 and a proximal portion 314 of the chamber 313. The vent needle 318 may include a penetration tip 327 disposed adjacent to a proximal end. The vent needle 318 may be disposed adjacent to a first or injection needle 317. The vent needle 318 can have a shorter length and extend a shorter distance into the proximal portion 314 than the injection needle 317.

In use, an ampule (not shown) can be disposed within the proximal portion 314 of the chamber 313 such that a septum of the ampule is punctured by both the injection needle 317 and the vent needle 318 and the needles 317, 318 are in fluid communication with a second medicament within the ampule. A distally directed force can be applied to the ampule such that the stopper 316 is displaced distally and a first medicament within a distal portion 315 of the chamber 313 is pressurized. When pressurized, a volume of the first medicament may flow through the injection needle 317 and into the ampule to compound or mix with the second medicament. Additionally, the second medicament is pressurized by the added volume of the first medicament. As a result, an equal volume of the second medicament to the injected volume of the first medicament may flow through the vent needle 318 and into the vent chamber 323 to prevent proximal displacement of a stopper of the ampule, as previously discussed. The second medicament vented into the vent chamber 323 may be absorbed by the absorbent mat 330 and/or flow through the vent channel 329.

As can be appreciated, various methods are also provided by the present disclosure. Examples include:

Example 1: A method of compounding medicaments, comprising: preparing a single dose vial comprising a first liquid medicament; inserting an ampule comprising a second liquid medicament into a chamber of the single dose vial; puncturing a septum of the ampule with a needle of the single dose vial; and injecting the first liquid medicament into the ampule to compound with the second liquid medicament, wherein the second liquid medicament is modified by the first liquid medicament.

Example 2. The method of Example 1, wherein the step of injecting the first liquid medicament comprises: applying a force to a stopper of the single dose vial with the ampule; displacing the stopper toward a closed end of the single dose vial; and displacing the first liquid medicament through the needle into the ampule.

Example 3. The method of Example 1, wherein a pH level of the second liquid medicament is brought closer to a pH of 7 when the first liquid medicament is compounded with the second liquid medicament.

Example 4. The method of Example 1, wherein the steps of the method of compounding liquid medicaments are performed at a point of care.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of compounding medicaments may include one or more of the following steps: preparing a single dose vial comprising a first liquid medicament; inserting an ampule comprising a second liquid medicament into a chamber of the single dose vial; puncturing a septum of the ampule with a needle of the single dose vial; injecting the first liquid medicament into the ampule to compound with the second liquid medicament; applying a force to a stopper of the single dose vial with the ampule; displacing the stopper toward a closed end of the single dose vial; and displacing the first liquid medicament through the needle into the ampule. Other steps are also contemplated.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment.

It will be appreciated that various features are sometimes grouped together in a single embodiment, figure, or description thereof for the purpose of streamlining the disclosure. Many of these features may be used alone and/or in combination with one another.

The phrases “coupled to” and “in communication with” refer to any form of interaction between two or more entities, including mechanical, electrical, magnetic, electromagnetic, fluid, and thermal interaction. Two components may be coupled to or in communication with each other even though they are not in direct contact with each other. For example, two components may be coupled to or in communication with each other through an intermediate component.

The directional terms “distal” and “proximal” are given their ordinary meaning in the art. That is, the distal end of a medical device means the end of the device furthest from the practitioner during use. The proximal end refers to the opposite end, or the end nearest to the practitioner during use. As specifically applied to a medicament compounding vial of this disclosure, the proximal end of the vial refers to the end nearest to the open end of the vial and the distal end refers to the opposite end, the end nearest to the closed end of the vial.

“Fluid” is used in its broadest sense, to refer to any fluid, including both liquids and gases as well as solutions, compounds, suspensions, etc., which generally behave as fluids.

References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially perpendicular” is recited with respect to a feature, it is understood that in further embodiments, the feature can have a precisely perpendicular configuration.

The terms “a” and “an” can be described as one, but not limited to one. For example, although the disclosure may recite a housing having “a stopper,” the disclosure also contemplates that the housing can have two or more stoppers.

Unless otherwise stated, all ranges include both endpoints and all numbers between the endpoints.

Recitation in the claims of the term “first” with respect to a feature or element does not necessarily imply the existence of a second or additional such feature or element.

The claims following this written disclosure are hereby expressly incorporated into the present written disclosure, with each claim standing on its own as a separate embodiment. This disclosure includes all permutations of the independent claims with their dependent claims. Moreover, additional embodiments capable of derivation from the independent and dependent claims that follow are also expressly incorporated into the present written description.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A vial for compounding liquid medicaments, comprising:
a housing comprising:
an open end;
a closed end; and
a chamber disposed between the open end and the closed end;
a removable cap disposed adjacent to the open end and configured to seal the chamber;
a displaceable stopper disposed within the chamber adjacent to the closed end;
an injection needle passing through the stopper and extending within the chamber from the stopper toward the open end, wherein the needle comprises a penetrating end disposed within the chamber; and
a first liquid medicament retained within the housing between the stopper and the closed end.

2. The vial of claim 1, wherein the vial is configured as a single dose or single use vial.

3. The vial of claim 1, wherein the housing is configured to receive an ampule filled with a second liquid medicament.

4. The vial of claim 1, wherein the first liquid medicament is sodium bicarbonate.

5. The vial of claim 1, wherein the first liquid medicament is any one of clonidine, dexmedetomidine, buprenorphine, dexamethasone, tramadol, midazolam, magnesium, epinephrine, morphine, fentanyl, and any combination thereof.

6. The vial of claim 1, wherein the first liquid medicament is sodium bicarbonate including a concentration ranging from 1.0% to 10.0%.

7. The vial of claim 1, wherein a volume of the first liquid medicament ranges from 0.1 ml to 1.0 ml.

8. The vial of claim 1, wherein a volume of the first liquid medicament is 0.15 ml.

9. The vial of claim 1, wherein the second liquid medicament is an anesthetic and is selected from the group of lidocaine, articaine, bupivacaine, carbocaine, marcaine, mepivacaine, prilocaine, and any combination thereof.

10. The vial of claim 1, wherein the second liquid medicament is any one of clonidine, dexmedetomidine, buprenorphine, dexamethasone, tramadol, midazolam, magnesium, epinephrine, morphine, fentanyl, and any combination thereof.

11. The vial of claim 1, wherein the penetrating end of the needle is disposed within the chamber at a position to prevent an accidental needle stick to a user.

12. The vial of claim 1, wherein at least a portion of the cap comprises a compliant material.

13. The vial of claim 12, wherein the compliant material is any one of a silicone, a rubber, and a thermal plastic elastomer.

14. The vial of claim 1, wherein the housing further comprises a flange disposed adjacent to the closed end.

15. The vial of claim 1, wherein the housing further comprises:
a vent chamber disposed adjacent to the closed end; and
a vent needle in fluid communication with the chamber and the vent chamber.

16. A system for compounding liquid medicaments, comprising:
a vial comprising:
a housing comprising:
an open end;
a closed end; and
a chamber disposed between the open end and the closed end;
a removable cap disposed adjacent to the open end and configured to seal the chamber;
a displaceable stopper disposed within the chamber adjacent to the closed end, the displaceable stopper defining a dispensing portion of the chamber that is on a side of the displaceable stopper toward the open end and a medicament portion of the chamber that is on an opposing side of the displaceable stopper toward the closed end;
a hollow needle passing through the stopper and extending from the medicament portion of the chamber toward the open end, wherein the needle extends from the displaceable stopper into the dispensing portion of the chamber; and
a first liquid medicament retained within the medicament portion of the chamber within the housing between the stopper and the closed end; and
an ampule comprising a second liquid medicament.

17. The injection system of claim 16,
wherein the ampule comprises a septum;
wherein the ampule is receivable within the chamber;
wherein the septum is puncturable by a penetrating end of the needle; and
wherein the first liquid medicament is injectable into the ampule through the needle.

18. The injection system of claim 16, further comprising a re-useable ampule holder.

19. The injection system of claim 16, wherein the vial is configured as a single dose or single use vial.
